(19) United States
(12) Patent Application Publication
(10) Pub. No.: US 2012/0143068 A1
(43) Pub. Date: Jun. 7, 2012

(54) COMPUTERIZE HEALTH MANAGEMENT METHOD AND HEALTH MANAGEMENT ELECTRONIC DEVICE

(76) Inventors: Chih-Chao CHENG, Zhonghe City (TW); Jung-Ping Chen, Sanchong City (TW)

(21) Appl. No.: 13/049,640

(22) Filed: Mar. 16, 2011

(30) Foreign Application Priority Data
Dec. 6, 2010 (TW) 99142457

(51) Int. Cl. A61B 5/021

(52) U.S. Cl. 600/485

(57) ABSTRACT

A computerized health management method and a health management electronic device are provided. The computerized health management method includes the following steps. A blood pressure curve of a user is measured. A systolic pressure and a diastolic pressure of the blood pressure curve are calculated by a microprocessor. A high blood pressure risk level of the user is analyzed by the microprocessor according to the systolic pressure and the diastolic pressure. A cardiovascular disease risk level of the user is analyzed by the microprocessor according to the high blood pressure risk level. A measuring frequency for measuring the blood pressure is suggested to the user by the microprocessor according to the high blood pressure risk level or the cardiovascular disease risk level.

This application claims the benefit of Taiwan application Serial No. 099142457, filed Dec. 6, 2010, the subject matter of which is incorporated herein by reference.

### BACKGROUND

1. Technical Field

The disclosure relates in general to a computerized health management method and a health management electronic device, and more particularly to a computerized health management method by way of measuring the blood pressure and a health management electronic device.

2. Description of the Related Art

Cardiovascular diseases such as heart diseases, arteriosclerosis, myocardial infarction, and stroke have long been a great threat to humans' health. From the view pint of medicine, a good understanding of the heart, blood pressure and blood vessels is directly related to the prevention of cardiovascular disease in addition to a good watch of proper diet and suitable exercise.

In terms of the evaluation of blood vessels, the blood pressure can be measured by a non-invasive way. However, the currently available device for evaluating the blood pressure can only passively provide measuring function and measured data. The user still cannot relate the measurement of the blood pressure to the risk level of self health, and does not know measuring time or the measuring period. Thus, the user still needs to visit the doctor in person periodically, which is indeed a waste of medical resources. Furthermore, the user cannot perform instant adjustment either.

### SUMMARY

The disclosure is directed to a computerized health management method and a health management electronic device.

According to a first aspect of the present disclosure, a computerized health management method is provided. The computerized health management method includes the following steps. A blood pressure curve of a user is measured. A systolic pressure and a diastolic pressure of the blood pressure curve are calculated by a microprocessor. A high blood pressure risk level of the user is analyzed by the microprocessor according to the systolic pressure and the diastolic pressure. A cardiovascular disease risk level of the user is analyzed by the microprocessor according to the high blood pressure risk level. A measuring frequency for measuring the blood pressure is suggested to the user by the microprocessor according to the high blood pressure risk level or the cardiovascular disease risk level.

According to a second aspect of the present disclosure, a health management electronic device is provided. The health management electronic device includes a measuring module and a microprocessor. The measuring module is for measuring a blood pressure curve of a user. The microprocessor includes a calculation unit, an analysis unit and a suggestion unit. The calculation unit calculates a systolic pressure and a diastolic pressure of the blood pressure curve and the number of cardiovascular disease risk factors which the user has. The analysis unit analyzes a high blood pressure risk level of the user according to the systolic pressure and the diastolic pressure, and analyzes a cardiovascular disease risk level according to the high blood pressure risk level. The suggestion unit suggests a measuring frequency for measuring the blood pressure to the user according to the high blood pressure risk level or the cardiovascular disease risk level.

The above and other aspects of the disclosure will become better understood with regard to the following detailed description of the non-limiting embodiment(s). The following description is made with reference to the accompanying drawings.

### BRIEF DESCRIPTION OF THE DRAWINGS

FIG. 1 shows a block diagram of a health management electronic device according to a first embodiment;

FIG. 2 shows a flowchart of a computerized health management method according to a first embodiment;

FIG. 3 shows a blood pressure curve;

FIG. 4 shows a block diagram of a health management electronic device according to a second embodiment;

FIGS. 5A-5B a flowchart of a computerized health management method according to a second embodiment;

FIG. 6 shows another blood pressure curve;

FIG. 7 shows a decision tree; and

FIG. 8 shows another decision tree.

### DETAILED DESCRIPTION

### First Embodiment

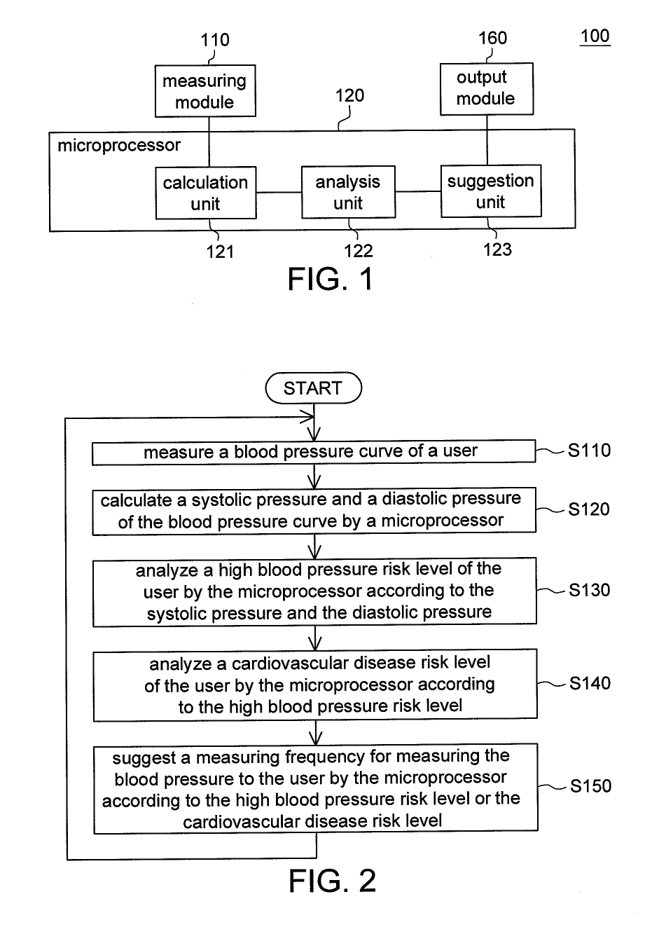

Referring to FIG. 1, a block diagram of a health management electronic device 100 according to a first embodiment is shown. The health management electronic device 100 includes a measuring module 110, a microprocessor 120 and an output module 160. The measuring module 110, used for measuring a blood pressure of the user, can be realized by such as an arm-type blood sphygmomanometer, a wrist type blood sphygmomanometer or a finger type blood sphygmomanometer. The microprocessor 120 includes a calculation unit 121, an analysis unit 122 and a suggestion unit 123. The calculation unit 121 is for executing various calculation processes. The analysis unit 122 is for executing various analysis processes. The suggestion unit 123 is for providing various suggestion information. The microprocessor 120 is realized by such as a micro-processing chip, a firmware circuit or a storage capable of storing several programs. The output module 160, used for outputting various information, can be realized by such as a display monitor or a loudspeaker. In the present embodiment of the disclosure, the measuring module 110 and the microprocessor 120 are directly disposed on the same electronic device, and signals are transmitted and communicated in the same electronic device not by way of remote-end connection.

Referring to FIG. 2, a flowchart of a computerized health management method according to a first embodiment is shown. Here below, the computerized health management method of the present embodiment of the disclosure is exemplified with the health management electronic device 100 of FIG. 1. However, the exemplification of the computerized health management method of the present embodiment of the disclosure is not limited to the health management electronic device of FIG. 1, and the sequence of steps in the exemplification of the computerized health management method of the present embodiment of the disclosure is not limited to that illustrated in FIG. 2.

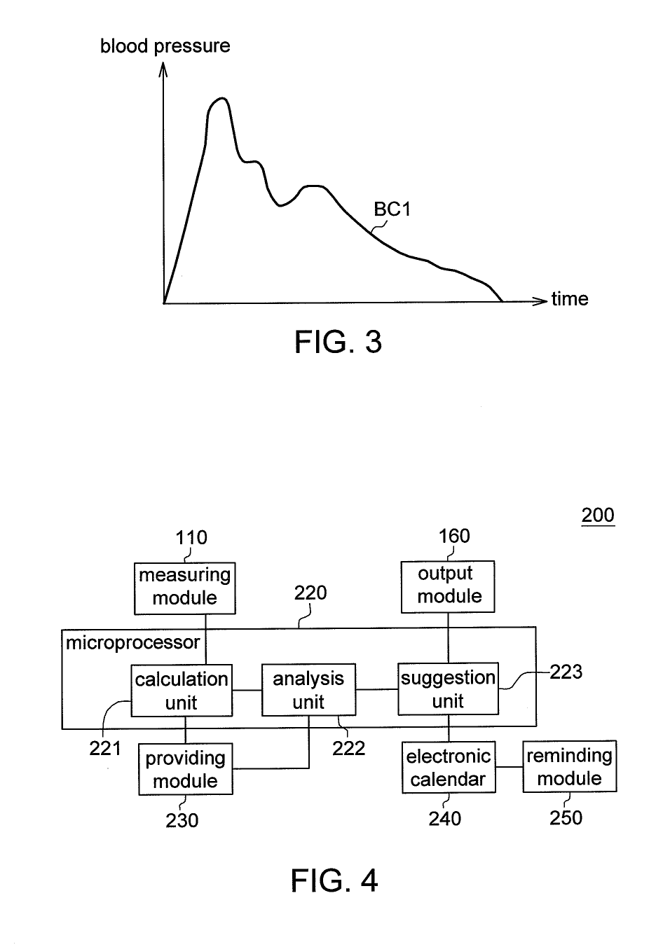

In step S110, a blood pressure curve BC1 of the user is measured by the measuring module 110, wherein the blood pressure curve BC1 is illustrated in FIG. 3

In step S120, a systolic pressure and a diastolic pressure of the blood pressure curve BC1 are calculated by the calculation unit 121 of the microprocessor 120. In the present embodiment of the disclosure, a typical periodic wave of the blood pressure curve BC1 is captured by the calculation unit 121 first, and then the systolic pressure and the diastolic pressure are calculated according to the typical periodic wave.

In step S130, a high blood pressure risk level of the user is analyzed by the analysis unit 122 of the microprocessor 120 according to the systolic pressure and the diastolic pressure. Referring to Table 1, the relationships between the systolic/diastolic pressure and the high blood pressure risk level are illustrated. The higher the systolic pressure and the diastolic pressure are, the higher the high blood pressure risk level of the user analyzed by the analysis unit 122 will be.

TABLE 1

| High Blood Pressure | | | | |
|---|---|---|---|---|
| Risk Level | Systolic Pressure | | Diastolic Pressure | |
| Normal | <120 | mmHg | <80 | mmHg |
| Early Stage High Blood | 120~139 | mmHg | 80~89 | mmHg |
| Pressure | | | | |
| 1^{st} Stage High Blood | 140~159 | mmHg | 90~99 | mmHg |
| Pressure (Minor) | | | | |
| 2^{nd} Stage High Blood | 160~179 | mmHg | 100~109 | mmHg |
| Pressure (Medium) | | | | |
| 3^{rd} Stage High Blood | ≧180 | mmHg | ≧110 | mmHg |
| Pressure (Severe) | | | | |

In step S140, a cardiovascular disease risk level of the user is analyzed by the analysis unit 122 of the microprocessor 120 according to the high blood pressure risk level. Referring to Table 2, the relationships between the high blood pressure risk level and the cardiovascular disease risk level are illustrated. When the consideration is limited to the high blood pressure risk level only, the analysis unit 122 will analyze the cardiovascular disease risk level of the user according to Table 2. The higher the high blood pressure risk level of the user is, the higher the cardiovascular disease risk level of the user analyzed by the analysis unit 122 will be.

TABLE 2

| | High Blood Pressure Risk Level | | | | |
|---|---|---|---|---|---|
| | | Early Stage | 1^{st} Stage | 2^{nd} Stage | 3^{rd} Stage |
| | | High | High | High | High |
| | | Blood | Blood | Blood | Blood |
| | Normal | Pressure | Pressure | Pressure | Pressure |
| Cardio- | Low | Low | Low | Medium | High |
| vascular | Risk | Risk | Risk | Risk | Risk |
| Disease | | | | | |
| Risk | | | | | |
| Level | | | | | |

In step S150, a measuring frequency for measuring the blood pressure is suggested to the user by the suggestion unit 123 of the microprocessor 120 according to the high blood pressure risk level or the cardiovascular disease risk level.

Referring to Table 3, the relationships between the high blood pressure risk level and the measuring frequency are illustrated. When the high blood pressure risk level is higher, the suggestion unit 123 will suggest a higher measuring frequency to the user. When the high blood pressure risk level is lower, the suggestion unit 123 will suggest a lower measuring frequency to the user.

TABLE 3

| High Blood Pressure | Measuring | |
|---|---|---|
| Risk Level | Frequency | Measuring Time |
| Normal | Monthly | Within 30 minutes after getting up |
| | | on one day of each month; |
| Early Stage High | Weekly | Within 30 minutes after up on one |
| Blood Pressure | | day of each week; |
| 1^{st} Stage High Blood | Daily | Within 30 minutes after getting up |
| Pressure (Minor) | | every day; |
| 2^{nd} Stage High Blood | Twice | Within 30 minutes after getting up |
| Pressure (Medium) | a Day | and before having dinner every day; |
| 3^{rd} Stage High Blood | 3 Times | Within 30 minutes after getting up, |
| Pressure (Severe) | a Day | before having dinner, and before |
| | | going to sleep every day; |

Referring to Table 4, the relationships between the cardiovascular disease risk level and the measuring frequency are illustrated. When the cardiovascular disease risk level is higher, the suggestion unit 123 will suggest a higher measuring frequency to the user. When the cardiovascular disease risk level is lower, the suggestion unit 123 will suggest a lower measuring frequency to the user.

TABLE 4

| Cardiovascular | | |
|---|---|---|
| Disease | Measuring | |
| Risk Level | Frequency | Measuring Time |
| Low Risk | Once a day | Within 30 minutes after getting up every |
| | | day; |
| Medium Risk | Twice | Within 30 minutes after getting up and |
| | a day | before having dinner every day; |
| High Risk | 3 Times | Within 30 minutes after getting up, before |
| | a day | having dinner, and before going to sleep |
| | | every day; |

After receiving the measuring frequency, the output module 160 will inform the user via display or voice and ask the user to follow the instruction.

Each time when the user measures blood pressure, the above steps S110˜S150 will be repeated. When the high blood pressure risk level or the cardiovascular disease risk level increases, the suggestion unit 123 will suggest the user to increase measuring frequency accordingly. When the high blood pressure risk level or the cardiovascular disease risk level decreases, the suggestion unit 123 will suggest the user to reduce measuring frequency. Thus, self health management can be performed via continuous measurement of blood pressure.

All the actions of steps S110˜S150 are executed by the health management electronic device 100. That is, the measurement, calculation, analysis and suggestion of blood pressure are all executed within the health management electronic device 100, not via any remote-end server or remote-end medical unit. The user can instantly obtain the suggestion of measuring frequency by the health management electronic device 100 without waiting for the analysis to be done by a remote-end server or a doctor.

### Second Embodiment

Referring to FIG. 4, a block diagram of a health management electronic device 200 according to a second embodiment is shown. The health management electronic device 200 of the present embodiment of the disclosure is different from the health management electronic device 100 of the first embodiment in that the health management electronic device 200 further includes a providing module 230, an electronic calendar 240 and a reminding module 250. The providing module 230, used for providing various information to the user, can be realized by such as a key board, a press key, a mouse, a touch panel, a measuring apparatus, a connection port for connecting a storage medium or a network card for connecting a network transmission information. When the providing module 230 is realized by a key board, a press key, a mouse or a touch panel, the information of the user can be provided by way of inputting. When the providing module 230 is realized by a measuring apparatus, the information of the user can be provided by way of measuring. When the providing module 230 is a connection port for connecting a storage medium, the information of the user can be directly retrieved from the storage medium. When the providing module 230 is realized by a network card, the information of the user can be directly obtained via a cabled or a wireless network transmission. The electronic calendar 240, used for recording a calendar of the user, can be realized by a combination of a micro-processing chip and a storage medium. The reminding module 250, used for emitting various signals to remind the user various events, can be realized by such as an alarm, a loudspeaker, a vibrator or a light emitter.

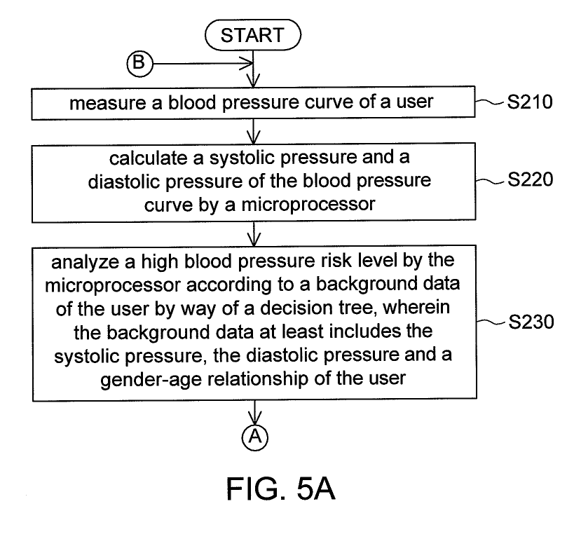

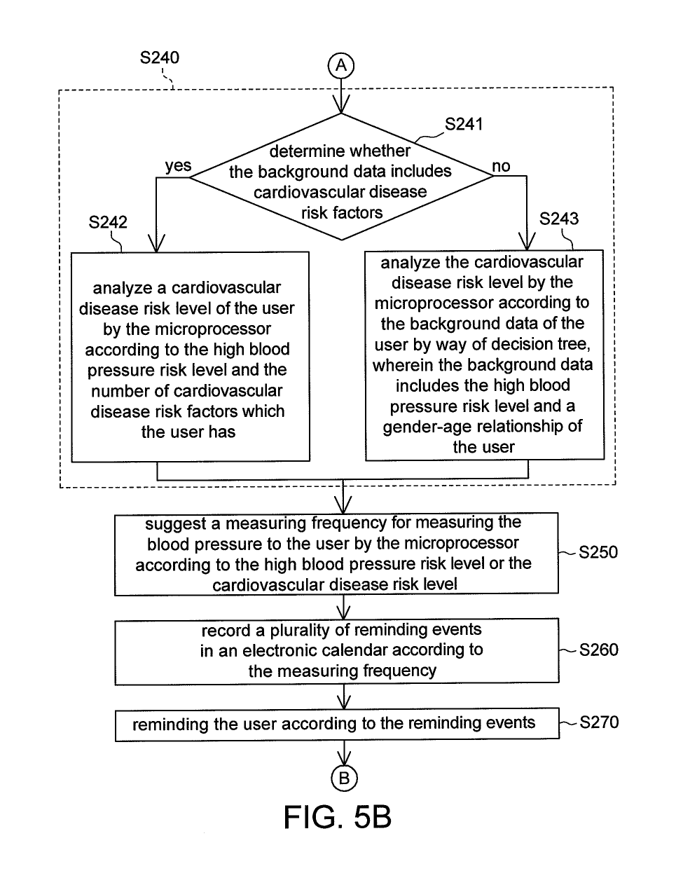

Referring to FIGS. 5A-5B, a flowchart of a computerized health management method according to a second embodiment is shown. The computerized health management method of the present embodiment of the disclosure is different from the computerized health management method of the first embodiment in that the computerized health management method of the present embodiment of the disclosure further includes steps S260 and S270, and more information are considered in step S230 of analyzing high blood pressure risk level and step S240 of analyzing cardiovascular disease risk level. Here below, the computerized health management method of the present embodiment of the disclosure is exemplified with the health management electronic device 200 of FIG. 4. However, the exemplification of the computerized health management method of the present embodiment of the disclosure is not limited to the health management electronic device 200 of FIG. 4, and the sequence of steps in the exemplification of the computerized health management method of the present embodiment of the disclosure is not limited to that illustrated in FIGS. 5A-5B.

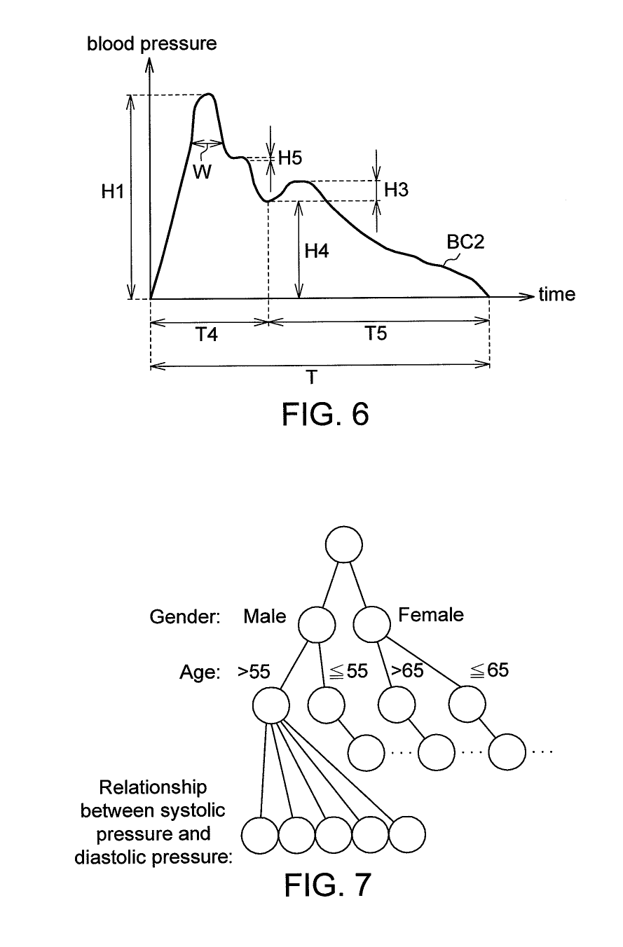

In step S210, a blood pressure curve BC2 of the user is measured by the measuring module 110, wherein the blood pressure curve BC2 is illustrated in FIG. 6

In step S220, a systolic pressure and a diastolic pressure of the blood pressure curve BC2 are calculated by the calculation unit 221 of the microprocessor 220. In the present step, the systolic pressure and the diastolic pressure are calculated by the calculation unit 221 by an adaptive characteristic ratio algorithm. As indicated in FIG. 6, the information of the adaptive characteristic ratio algorithm includes at least one of a cardiac cycle T, a systolic period T4, a diastolic period T5, a width at ⅓ of primary wave W, a primary wave height H1, a tidal wave height H5, a dicrotic notch height H4 and a dicrotic wave height H3 of the blood pressure curve BC2.

In the step S220 of an embodiment, the calculation unit 221 can further calculate the systolic pressure and the diastolic pressure by a wavelet algorithm, wherein the information of the wavelet algorithm includes at least one of an amplitude, a width, an area, a slope, a ratio of the blood pressure curve BC2.

Then, the method proceeds to step S230, a high blood pressure risk level is analyzed by the analysis unit 222 of the microprocessor 220 according to a background data of the user by way of a decision tree, wherein the background data at least includes the systolic pressure, the diastolic pressure and a gender-age relationship of the user. The contents of the background data are mostly provided by the providing module 230. Referring to FIG. 7, a decision tree of step S204 is shown. The first to the second layer of the decision tree indicate the gender and the age of the user, and the third layer of the decision tree indicates the relationship between the systolic pressure and the diastolic pressure of Table 1. The analysis unit 222 analyzes the high blood pressure risk level of the user layer by layer. The background data of FIG. 7 constructs a three-layer decision tree with gender, age, and the relationship between the systolic pressure and the diastolic pressure. In other embodiments, more background data can be used for constructing a decision tree of more than three layers (or even tens of layers) for analyzing the high blood pressure risk level.

In step S240, the cardiovascular disease risk level of the user is analyzed by the analysis unit 122 of the microprocessor 120 according to the high blood pressure risk level, wherein step S240 includes sub-steps S241˜S243. In step S240 of an embodiment, the cardiovascular disease risk level is analyzed according to the high blood pressure risk level, and several cardiovascular disease risk factors of the background data of the user also can further be included for analyzing the cardiovascular disease risk level (disclosed in step S241). In step 240 of another embodiment, the decision tree can be used for determining the cardiovascular disease risk factors.

In step S241, whether the background data includes cardiovascular disease risk factors is determined by the analysis unit 122, wherein part of the contents of the background data can be provided by the supply module 230. If the background data includes cardiovascular disease risk factors, then the method proceeds to step S242. If the cardiovascular disease risk factors cannot be determined according to the background data of the user, then the method proceeds to step S243.

In step S242, the cardiovascular disease risk level of the user is analyzed by the analysis unit 122 of the microprocessor 120 according to the high blood pressure risk level and the number of cardiovascular disease risk factors which the user has. Referring to Table 5, various cardiovascular disease risk factors are illustrated. When the user has these cardiovascular disease risk factors, this implies that the user is at the risk of cardiovascular diseases.

TABLE 5

| | Cardiovascular Disease Risk Factors |
|---|---|
| 1 | Male aged above 55 |
| 2 | Female aged above 65 |
| 3 | Smoker |
| 4 | Total cholesterol (TC) >240 mg/dl, or low-density lipoprotein |
| | cholesterol (LDL) >160 mg/dl |
| 5 | Family has early onset of cardiovascular disease and aged under 50; |
| 6 | Male whose high-density lipoprotein (HDL) <40 mg/dl |
| 7 | Female whose high-density lipoprotein (HDL) <45 mg/dl |
| 8 | Obese or body mass index (BMI) ≧27 |
| 9 | Exercise averter |

Referring to Table 6, the relationships between the high blood pressure risk level, the number of cardiovascular disease risk factors and the cardiovascular disease risk level are illustrated. The research shows that the higher the high blood pressure risk level of the user is, the higher the cardiovascular disease risk level will be; the more the number of the cardiovascular disease risk factors is, the higher the cardiovascular disease risk level will be. Let Table 6 be taken for example. The factors of analysis include high blood pressure risk level and the number of other cardiovascular disease risk factors. After the blood pressure of the user is measured, if the high blood pressure risk level is rated as “the first stage high blood pressure” and the number of cardiovascular disease risk factors is “2”, then the analysis unit 222 rates the cardiovascular disease risk level of the user as “medium risk”. After the blood pressure of the user is measured, if the high blood pressure risk level is rated as “the second stage high blood pressure” and the number of cardiovascular disease risk factors is “3”, then the analysis unit 222 rates the cardiovascular disease risk level of the user as “high risk”.

TABLE 6

| | High Blood Pressure Risk Level | | |
|---|---|---|---|
| | 1^{st} Stage | 2^{nd} Stage | 3^{rd} Stage |
| Number of Cardio- | High Blood | High Blood | High Blood |
| vascular Disease | Pressure | Pressure | Pressure |
| Risk Factors | Cardiovascular Disease Risk Level | | |
| 0 | Low Risk | Medium Risk | High Risk |
| 1~2 | Medium Risk | Medium Risk | High Risk |
| 3 and above | High Risk | High Risk | High Risk |

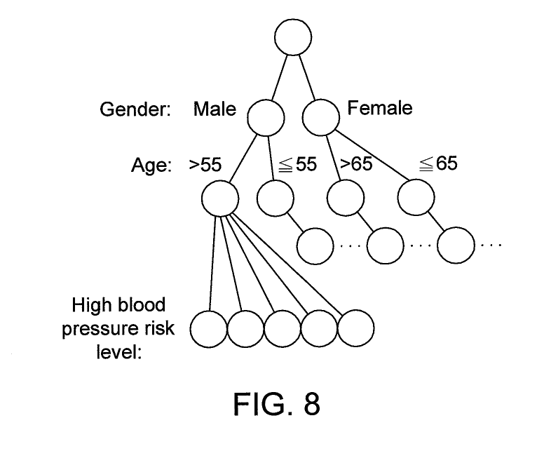

In step S243, the cardiovascular disease risk level is analyzed by the analysis unit 122 of the microprocessor 120 according to the background data of the user by way of decision tree, wherein the background data includes a high blood pressure risk level and a gender-age relationship of the user. Referring to FIG. 8, a decision tree of step S243 is shown. In the present embodiment of the disclosure, the cardiovascular disease risk level is analyzed by the analysis unit 222 according to the background information of the user by way of decision tree. Let FIG. 8 be taken for example. The first to the second layer of decision tree indicate the gender and the age of the user, and the third layer of the decision tree indicates the classification of the high blood pressure risk level. The analysis unit 222 analyzes the cardiovascular disease risk level of the user by filtering the decision tree layer by layer. The background data of FIG. 8 constructs a three-layer decision tree with the gender-age relationship and the high blood pressure risk level of the user only. In other embodiments, more background data can be used for constructing a decision tree of more than three layers (or even tens of layers) for analyzing the cardiovascular disease risk level. Following that, the method proceeds to step S250, a measuring frequency for measuring the blood pressure is suggested to the user by the suggestion unit 223 of the microprocessor 220 according to the high blood pressure risk level or the cardiovascular disease risk level. Meanwhile, the suggestion unit 223 will suggest whether the user should go to see a doctor according to the high blood pressure risk level and the cardiovascular disease risk level.

In step S260, a number of reminding events are recorded in the electronic calendar 240 according to the measuring frequency.

In step S270, the reminding module 250 reminds the user according to these reminding events.

Each time when the user measures the blood pressure, the above steps will be repeated. When the high blood pressure risk level or the cardiovascular disease risk level changes, the suggestion unit 223 will change the contents of suggestion accordingly. Thus, self health management can be performed via continuous measurement of blood pressure

While the disclosure has been described by way of example and in terms of the exemplary embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A computerized health management method, comprising:
measuring a blood pressure curve of a user;
calculating a systolic pressure and a diastolic pressure of the blood pressure curve by a microprocessor;
analyzing a high blood pressure risk level of the user by the microprocessor according to the systolic pressure and the diastolic pressure;
analyzing a cardiovascular disease risk level of the user by the microprocessor according to the high blood pressure risk level; and
suggesting a measuring frequency for measuring the blood pressure to the user by the microprocessor according to the high blood pressure risk level or the cardiovascular disease risk level.

2. The computerized health management method according to claim 1, wherein in the step of analyzing the high blood pressure risk level, the microprocessor analyzes the high blood pressure risk level according to a background data of the user by way of a decision tree, and the background data comprises the systolic pressure and the diastolic pressure and a gender-age relationship of the user.

3. The computerized health management method according to claim 1, wherein in the step of calculating the systolic pressure and the diastolic pressure, the microprocessor calculates the systolic pressure and the diastolic pressure by an adaptive characteristic ratio algorithm, and the information of the adaptive characteristic ratio algorithm comprises at least one of a cardiac cycle, a systolic period, a diastolic period, a width at ⅓ of primary wave, a primary wave height, a tidal wave height, a dicrotic notch height and a dicrotic wave height of the blood pressure curve.

4. The computerized health management method according to claim 1, wherein in the step of calculating the systolic pressure and the diastolic pressure, the microprocessor calculates the systolic pressure and the diastolic pressure by a wavelet algorithm, and the information of the wavelet algorithm comprises at least one of an amplitude, a width, an area, a slope, a ratio of the blood pressure curve.

5. The computerized health management method according to claim 1, further comprising:
recording a plurality of reminding events in an electronic calendar according to the measuring frequency; and
reminding the user according to the reminding events.

6. The computerized health management method according to claim 1, wherein in the step of analyzing the cardiovascular disease risk level of the user, the microprocessor analyzes the cardiovascular disease risk level of the user according to the high blood pressure risk level and the number of cardiovascular disease risk factors which the user has.

7. The computerized health management method according to claim 1, wherein in the step of analyzing the cardiovascular disease risk level, the microprocessor analyzes the cardiovascular disease risk level according to a background data of the user by way of a decision tree, and the background data comprises the high blood pressure risk level and a gender-age relationship of the user.

8. A health management electronic device, comprising:
a measuring module for measuring a blood pressure curve of a user; and
a microprocessor, comprising:
a calculation unit for calculating a systolic pressure of the blood pressure curve and a diastolic pressure of the blood pressure curve;
an analysis unit for analyzing a high blood pressure risk level of the user according to the systolic pressure and the diastolic pressure and analyzing a cardiovascular disease risk level of the user according to the high blood pressure risk level; and
a suggestion unit for suggesting a measuring frequency for measuring the blood pressure to the user according to the high blood pressure risk level or the cardiovascular disease risk level.

9. The health management electronic device according to claim 8, wherein the analysis unit further analyzes the high blood pressure risk level according to a background data of the user by way of a decision tree, and the background data comprises the systolic pressure, the diastolic pressure and a gender-age relationship of the user.

10. The health management electronic device according to claim 8, wherein the calculation unit calculates the systolic pressure and the diastolic pressure by an adaptive characteristic ratio algorithm, and the information of the adaptive characteristic ratio algorithm comprises at least one of a cardiac cycle, a systolic period, a diastolic period, a width at ⅓ of primary wave, a primary wave height, a tidal wave height, a dicrotic notch height and a dicrotic wave height of the blood pressure curve.

11. The health management electronic device according to claim 8, wherein the calculation unit calculates the systolic pressure and the diastolic pressure by a wavelet algorithm, and the information of the wavelet algorithm comprises at least one of an amplitude, a width, an area, a slope, a ratio of the blood pressure curve.

12. The health management electronic device according to claim 8, further comprising:
an electronic calendar for recording a plurality of reminding events according to the measuring frequency; and
a reminding module for reminding the user according to the reminding events.

13. The health management electronic device according to claim 8, wherein the analysis unit analyzes the cardiovascular disease risk level of the user according to the high blood pressure risk level and the number of cardiovascular disease risk factors which the user has.

14. The computerized health management device according to claim 8, wherein the analysis unit further analyzes the cardiovascular disease risk level according to a background data of the user by way of a decision tree, and the background data comprises the high blood pressure risk level and a gender-age relationship of the user.
